(19) United States
(12) Patent Application Publication
(10) Pub. No.: US 2008/0075785 A1
(43) Pub. Date: Mar. 27, 2008

(54) CONTROLLED RELEASE HYDROGEL FORMULATION

(76) Inventors: San-Laung CHOW, San Jose, CA (US); David Wong, Milpitas, CA (US); Damian Garcia, Sunnyvale, CA (US)

Correspondence Address: PATTERSON & SHERIDAN, L.L.P., 3040 POST OAK BOULEVARD, SUITE 1500, HOUSTON, TX, 77056, US

(21) Appl. No.: 11/772,017

(22) Filed: Jun. 29, 2007

Related U.S. Application Data
(60) Provisional application No. 60/826,728, filed on Sep. 22, 2006.

(51) Int. Cl. A61K 9/14; A61K 31/47; A61P 43/00; A61K 31/496

(52) U.S. Cl. 424/499; 424/489; 514/252.17; 514/312

(57) ABSTRACT

Embodiments of the invention generally provide pharmaceutical drug compositions, methods of preparing oral drug compositions, such as controlled release dosage compositions for hydrophobic active ingredients. In one aspect, the invention provides a pharmaceutical formulation comprising a therapeutically effective amount of a hydrophobic drug, an adjustable ratio of a non-cross linked hydrogel polymer and a non-gelling insoluble polymer. One example is a controlled release pharmaceutical composition which includes 1% to 80% of a therapeutically amount of cilostazol, 4% to 80% of a water-swelling hydrogel polymer, and 4% to 80% of a non-gelling insoluble polymer. In another aspect, a constant release profile of the pharmaceutical formulation is obtained. In another aspect, a zero degree release profile of the pharmaceutical formulation is obtained. Further, a method for treating intermittent claudication using the pharmaceutical formulation is provided.

### CROSS-REFERENCE TO RELATED APPLICATIONS

This application claims benefit of U.S. provisional patent application Ser. No. 60/826,728, filed Sep. 22, 2006, which is herein incorporated by reference.

### BACKGROUND OF THE INVENTION

The invention generally relates to pharmaceutical compositions, such as drug formulations present in a solid form for oral administration. More particularly, the invention relates to long-lasting sustained dosage compositions, and carriers and active ingredients in the compositions thereof, such as controlled release, sustained release, and extended release drug compositions for oral dosage formulations containing a drug and a carrier material.

Drug delivery at a predetermined rate such that drug concentrations can be maintained at desired therapeutically effective levels over an extended period, has received a great deal of attention. Many known solid drug formulations are required to be taken orally three or four times a day. There is a need for oral formulations to be taken less often, such as once per day. In addition, there are other problems with undesired drug delivery rate. For example, various side effects are observed for immediate release drug formulations due to high drug concentrations released in the plasma or blood stream right after the intake of the drug.

Many hydrophobic active ingredients present challenges in formulating into prolong release pharmaceutical compositions due to their poor aqueous solubility and slow dissolution rate during drug delivery. Micronization and emulsion have been proposed to enhance in vivo performance. However, these approaches have several disadvantages including stability, drug precipitation and packaging issues. Further, incorporating polymers to formulate sustained release pharmaceutical compositions for hydrophobic active ingredients have commonly exhibited an undesirable initial burst in their release profiles and resulted in less than optimal, non-constant and often non-linear release rate.

Therefore, there is a need for formulating improved controlled release formulations for hydrophobic active ingredients and method for preparing such a controlled release formulation.

### SUMMARY OF THE INVENTION

Embodiments of the invention generally provide pharmaceutical drug compositions, methods of preparing oral drug compositions, such as controlled release dosage compositions for hydrophobic active ingredients. In one embodiment, a pharmaceutical composition having one or more hydrogel materials or aqueous swelling polymers is provided to be combined with a release rate-adjusting polymer in a ratio for controlling the release rate of the hydrophobic active ingredients in vivo and in vitro.

In another embodiment, the pharmaceutical composition may include a therapeutically-effective amount of a powder form of a hydrophobic drug, a non-cross-linked, water-swelling homo-polymer, and a non-gelling insoluble polymer, where the non-cross-linked, water-swelling homo-polymer and the non-gelling insoluble polymer are combined at a weight ratio of about 1:10 to 10:1.

In another embodiment, a pharmaceutical composition includes a powder form of a non-cross-linked, water-swelling homo-polymer and a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of a hydrophobic drug.

In another embodiment, a controlled release pharmaceutical composition may include a powder form of a non-cross-linked, water-swelling homo-polymer and a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of cilostazol at about 1% to 95% by weight of the pharmaceutical composition.

In another embodiment, an controlled release pharmaceutical composition may include a powder form of a non-cross-linked, water-swelling homo-polymer and a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of doxazocin mesylate at about 1% to about 95% by weight of the pharmaceutical composition.

Further, a method for administering a pharmaceutical composition containing a therapeutically-effective amount of a powder form of a hydrophobic drug may include administering to a mammal an effective amount of the pharmaceutical composition comprising a power form of a non-cross-linked, water-swelling homo-polymer and a powder form of a non-gelling insoluble polymer, where the non-cross-linked, water-swelling homo-polymer and the non-gelling insoluble polymer are combined at a weight ratio of about 1:10 to 10:1 and directly compressed with the hydrophobic drug.

Still, further, a method for treating intermittent claudication using a pharmaceutical formulation is provided. The method may include administering to a mammal an effective amount of the pharmaceutical composition comprising a power form of a non-cross-linked, water-swelling homo-polymer and a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of cilostazol.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates exemplary release rate profiles for representative drug formulations in accordance with one embodiment of the invention.

FIG. 2 illustrates exemplary release rate profiles at different pH for representative tablets of a hydrophobic drug in accordance with one embodiment of the invention.

FIG. 3 illustrates an exemplary release rate profile for representative tablets of a hydrophobic drug prepared at different dosage strength and with different concentration of a wetting agent as compared to examples in FIG. 1 in accordance with one embodiment of the invention.

FIG. 4 illustrates an exemplary release rate profile for representative tablets of a hydrophobic drug prepared at different dosage strength as compared to the example in FIG. 3 in accordance with one embodiment of the invention.

FIG. 5 illustrates an exemplary release rate profile for representative tablets of a hydrophobic drug prepared with different polymers as compared to the example in FIG. 2 in accordance with one embodiment of the invention.

FIG. 6 illustrates an exemplary release rate profile for representative tablets of a hydrophobic drug prepared with different polymers as compared to the example in FIG. 5 in accordance with one embodiment of the invention.

FIG. 7 illustrates exemplary release rate profiles for representative tablets of cilostazol in accordance with one embodiment of the invention.

### DETAILED DESCRIPTION OF THE INVENTION

A pharmaceutical composition having at least one aqueous swelling hydrogel polymer materials is provided. In one embodiment, a hydrogel-based pharmaceutical dosage system that provides sustained release of a hydrophobic drug is obtained. According to one or more embodiments of the invention, the pharmaceutical composition is capable of providing a controlled release rate, such as a substantially zero-order release rate for hydrophobic active ingredients.

In one embodiment, a pharmaceutical composition for a hydrophobic drug may include a hydrogel material and a release rate-adjusting polymer in a ratio to achieve desired in vitro dissolution (and, consequently, in vivo bioavailability) performance. The ratio of the hydrogel material and the release rate-adjusting polymer can be, for example, a weight ratio of about 1:20 to 20:1, such as a weight ratio of about 1:10 to 10:1.

The release rate adjusting polymer may be, for example, a non-gelling insoluble polymer, a hydrophobic polymer, an enteric polymer, etc. In addition, an effective amount of a non-toxic, pharmaceutically acceptable stabilizing ionizable compound can be included to assist the hydrogel material and modify the release rate of the therapeutically active drug. The stabilizing ionizable compound may be, for example, a wetting agent, a surfactant (e.g., sodium lauryl sulfate, tween-20, tween-80, PEG, etc.), an excipient (e.g., diluents, binders, release modifying agents, glidants and lubricants, etc.), among others.

One example of a pharmaceutical formulation may include a therapeutically effective amount of a hydrophobic drug, a non-cross-linked, water-swelling homo-polymer hydrogel, and a non-gelling insoluble polymer. The hydrophobic drug as described herein generally includes active drug ingredients that are moderately, to poorly soluble in water, e.g., any organic or inorganic compound or substance having biological or pharmaceutical activity with room temperature water solubility of less than about 1 g/mL, such as less than 100 mg/ml, or having a log P greater than 2, or being lipid soluble, or not adsorbing water, etc.

For example, the hydrophobic drug may be a poorly water soluble pharmaceutically active compound intended for oral administration but does not generally dissolve easily and rapidly in the gastrointestinal tract. This hydrophobic property often makes it difficult to formulate a drug so that it exhibits a satisfactory bioavailability profile in vivo. Poor bioavailability may lead to ineffective therapy, the need for higher dosing and/or undesirable side effects. Exemplary compounds are provided herein. It will be appreciated that the room temperature water solubility for any given compound can be easily determined using readily available chemistry techniques and tools, such as high performance liquid chromatography or spectrophotometry.

The hydrophobic drugs, and their pharmaceutically acceptable salts thereof, which may be formulated in accordance with the present invention include, without limitation, the following: Analgesics and anti-inflammatory agents: acetaminophen, aloxiprin, auranofin, azapropazone, benorylate, celecoxib, diflunisal, etodolac, fenbufen, fenoprofen, flurbiprofen, ibuprofen, indomethacin, ketoprofen, meclofenamic acid, mefenamic acid, nabumetone, naproxen, oxyphenbutazone, phenylbutazone, piroxicam, rofecoxib, salicylamide, salicylic acid, sulindac; Anthelmintics: albendazole, bephenium hydroxynaphthoate, cambendazole, dichlorophen, ivermectin, mebendazole, oxamniquine, oxantel embonate, oxfendazole, praziquantel, pyrantel embonate, thiabendazole; Anti-arrhythmic agents: amiodarone, disopyramide, flecainide, quinidine; Anti-bacterial agents: benethamine, cefaclor, cinoxacin, ciprofloxacin, clarithromycin, clofazimine, cloxacillin, demeclocycline, doxycycline, erythromycin, ethionamide, imipenem, nalidixic acid, nitrofurantoin, penicillin, rifampicin, spiramycin, sulphabenzamide, sulphacetamide, sulphadiazine, sulphadoxine, sulphafurazole, sulphamerazine, sulphamethoxazole, sulphapyridine, tetracycline, trimethoprim; Anti-coagulants: dicoumarol, dipyridamole, nicoumalone, phenindione; Anti-depressants: amoxapine, maprotiline, mianserin, nortriptyline, oxypertine, trazodone, trimipramine, venlafaxine; Anti-diabetics: acetohexamide, chlorpropamide, glibenclamide, gliclazide, glipizide, tolazamide, tolbutamide; Anti-epileptics: beclamide, carbamazepine, clonazepam, ethotoin, metharbital, methoin, methsuximide, methylphenobarbitone, oxcarbazepine, paramethadione, phenacemide, phenobarbitone, phensuximide, phenytoin, primidone, sulthiame, valproic acid; Anti-fungal agents: amphotericin, butoconazole, clotrimazole, econazole, fluconazole, flucytosine, griseofulvin, itraconazole, ketoconazole, miconazole, natamycin, nystatin, sulconazole, terbinafine, terconazole, tioconazole, undecenoic acid; Anti-gout agents: allopurinol, probenecid, sulphinpyrazone; Anti-hypertensive agents: amlodipine, benidipine, darodipine, diazoxide, dilitazem, felodipine, guanabenz, isradipine, methyldopa, minoxidil, nicardipine, nifedipine, nimodipine, phenoxybenzamine, prazosin, reserpine, terazosin; Anti-malarials: amodiaquine, chloroquine, chlorproguanil, halofantrine, mefloquine, proguanil, pyrimethamine, quinine; Anti-migraine agents: dihydroergotamine, ergotamine, methysergide, pizotifen, sumatriptan; Anti-muscarinic agents: atropine, benzhexol, biperiden, ethopropazine, hyoscyamine, mepenzolate, oxyphencylcimine, tropicamide; Anti-neoplastic agents and immunosuppressants: aminoglutethimide, amsacrine, azathioprine, busulphan, chlorambucil, cyclosporin, dacarbazine, estramustine, etoposide, finasteride, lomustine, melphalan, mercaptopurine, methotrexate, mitomycin, mitotane, mitozantrone, procarbazine, raloxifene, tamoxifen, testolactone; Anti-Parkinsonian agents: bromocriptine, lysuride; Anti-protazoal agents: benznidazole, clioquinol, decoquinate, diiodohydroxyquinoline, diloxanide, dinitolmide, furzolidone, metronidazole, nimorazole, nitrofurazone, omidazole, tinidazole; Anti-thyroid agents: carbimazole, propylthiouracil; Anxiolytics, sedatives, hypnotics and neuroleptics: allobarbitone, allylbarbituric acid, alprazolam, amylobarbitone, barbitone, bentazepam, bromazepam, bromperidol, brotizolam, butobarbitone, carbromal, carphenazine, chlordiazepoxide, chlormethiazole, chlorpromazine, clobazam, clotiazepam, clozapine, cyclobarbitone, diazepam, droperidol, ethinamate, flunanisone, flunitrazepam, fluopromazine, flupenthixol, fluphenazine, flurazepam, haloperidol, lorazepam, lormetazepam, medazepam, meprobamate, methaqualone, midazolam, nitrazepam, oxazepam, pentobarbitone, perphenazine, pimozide, prochlorperazine, sulpiride, temazepam, thioridazine, triazolam, zopiclone; β-Blockers: acebutolol, alprenolol, atenolol, labetalol, metoprolol, nadolol, oxprenolol, pindolol, propranolol; Cardiac Inotropic agents: amrinone, digitoxin, digoxin, enoximone, lanatoside C, medigoxin; Corticosteroids: beclomethasone, betamethasone, budesonide, cortisone, desoxymethasone, dexamethasone, flucortolone, fludrocortisone, flunisolide, fluticasone, hydrocortisone, methylprednisolone, prednisolone, prednisone, triamcinolone; Diuretics: acetazolamide, amiloride, amisometradine, bendroflumethiazide, bumetanide, chlorothiazide, chlorthalidone, ethacrynic acid, furosemide, hydrochlorothiazide, metolazone, spironolactone, triamterene; Gastro-intestinal agents: aminosalicylic acid, bisacodyl, cimetidine, cisapride, diphenoxylate, domperidone, famotidine, loperamide, mesalazine, nizatidine, omeprazole, ondansetron, ranitidine, sulphasalazine; Histamine H.sub.1-Receptor Antagonists: acrivastine, astemizole, cinnarizine, cyclizine, cyproheptadine, dimenhydrinate, fexofenadine, flunarizine, loratadine, meclozine, oxatomide; Lipid-regulating agents: atorvastatin, bezafibrate, clofibrate, dextrothyroxine, fenofibrate, gemfibrozil, lovastatin, probucol, simvastatin, fibrates, fenofibrates; Nitrates and other anti-anginal agents: amyl nitrate, glyceryl trinitrate, isosorbide dinitrate, isosorbide mononitrate, pentaerythritol tetranitrate; Nutritional agents: betacarotene, vitamin A, vitamin B, vitamin D, vitamin E, vitamin K; Opioid analgesics: codeine, dextropropyoxyphene, diamorphine, dihydrocodeine, meptazinol, methadone, morphine, nalbuphine, pentazocine; Platelet aggregation inhibitors: cilostazol, clopidogrel, ticlopidine, dipyridamole, aspirin; Respiratory agents: montelukast, pranlukast (CCN00401), zafirlukast, zileuton; Sex hormones: clomiphene, conjugated estrogens, danazol, estradiol, ethinyloestradiol, medrogestone, medroxyprogesterone acetate, mestranol, methyltestosterone, norethisterone, norgestimate, norgestrel, progesterone, stanozolol, stiboestrol, testosterone, tibolone; Stimulants: amphetamine, cocaine, dexamphetamine, dexfenfluramine, fenfluramine, mazindol; Thyroid agents: levothyroxine, their pharmaceutically equivalent salts, among others.

Other biopharmaceutical compounds useful for the practice of the instant invention include, but are not limited to, sildenafil (VIAGRA™), acyclovir, gancyclovir, fexofenidine, celecoxib (CELEBREX™), rofecoxib (VIOXX™), androstenedione, chloroquine, diphenhydramine HCl, buspirone, doxazocin mesylate, loratadine, clomiphine, zinc gluconate, zinc acetate, hydrocortisone, warfarin, indinavir sulfate, lidocaine, novacaine, estradiol, norethindrone acetate, medroxyprogesterone, dexfenfluramine, dextroamphetamine, doxycycline, thalidomide, fluticasone, fludarabine phosphate, etanercept, metformin hydrochloride, hyaluronate, tetrazocin hydrochloride, loperamide, ibogaine, clonazepam, ketamine, lamivudine (3TC™), isotretinoin, nicotine, mefloquine, levofloxacin, atorvastatin (LIPITOR™), miconazole nitrate (MONISTAT™), ritonavir, famotidine, simvastatin (ZOCOR™), sibutramine HCl monohydride, ofloxacin, lansoprozole, raloxifene (EVISTA™), zanamivir (RELENZA™), oseltamivir phosphate, 4-phenylbutyric acid sodium salt, chlorpromazine, nevirapine, zidovudine, cetirizine hydrochloride (ZYRTEC™), bisphosphonates such as pamidronate and zoledronate, nifedipine, felodipine, their pharmaceutically equivalent salts, and the like.

One example of a hydrophobic drug is cilostazol or its pharmaceutically equivalent salts thereof. Another example of a hydrophobic drug is doxazocin mesylate or its pharmaceutically equivalent salts thereof. Cilostazol inhibits phosphdiesterase III and increases cyclic AMP in platelets, resulting in inhibition of platelet aggregation and vasodilation. Thus, cilostazol is indicated to be used therapeutically for intermittent claudication. Platelet aggregation inhibitors, such as Cilostazol, are used primarily to treat and prevent arterial thrombosis. Platelets play an important role in stopping hemorrhage caused by damage to blood vessel through aggregation to form thrombi When vascular endothelium is injured or the blood vessel is narrowed (e.g., during arteriosclerosis), platalets tend to aggregate and trigger thrombus or embolus formation, causing ischemic diseases, such as myocardia infarction, angina pectoris, ischemic cerebrovascular disorder, and peripheral vascular disease. Therefore, platelet aggregation inhibitors can be administered to a subject for prevention and treatment of related schemic dseases.

Other platelet aggregation inhibitors include salicylates, adenosine diphosphate (ADP) inhibitors, glycoprotein IIb/IIIa antagonists, platelet derived growth factor, indirect thrombin inhibitors, cAMP-phosphodiesterase inhibitors, and anti-inflammatory agents. Aspirin is the oldest antiplatelet agent and works via inhibition of cyclooxygenase. Dipyridamole inhibits the uptake of adenosine and increases the evels of cyclic AMP. AGGRENOX™, which combines dipyrdamole and aspirin, utilizes the different mechanisms of action of the two agents to inhibit platelet aggregation. Clopidogrel and ticlopidine inhibit the binding of adenosine diphosphate (ADP) to their piatelet receptors and subsequently inhibit platelet aggregation. The indications for clopidogrel and ticlopidine include secondary prevention of stroke, myocardial infarction, acute coronary syndrome or other vascular death. Other potential anti-platelet aggregation agents under studies or less common include nattokinase, lotrafiban, oprostenol, terocyclic-substituted tricyclics, abciximab, eptifibatide, beraprost (1H-Cyclopenta[b]benzofuran-5-butanoic acid, 2,3,3a,8b-tetrahydro-2-hydroxy-1-(3-hydroxy-4-methyl-1-octen-6-ynyl), acadesine (1H-imidazole-4-carboxamide, 5-amino-1-β-D-ribofuranosyl-), beraprost sodium (1H-cyclopenta[b]benzofuran-5-butanoic acid, 2,3,3a,8b-tetrahydro-2-hydroxy-1-(3-hydroxy-4-methyl-1-octen-6-ynyl)-, monosodium salt, ciprostene calcium (pentanoic acid, 5-[(3aS,5R,6R,6aR)-hexahydro-5-hydroxy-6-[(1E,3S)-3-hydroxy-1-octenyl]-3a-methyl-2(1H)-pentalenylidene]-, calcium salt (2:1), (5Z)-), itazigrel (thiazole, 4,5-bis(4-methoxyphenyl)-2-(trifluoromethyl)), lifarizine (piperazine, 1-(diphenylmethyl)-4-[[5-methyl-2-(4-methylphenyl)-1H-imidazol-4-yl]methyl]-), oxagrelate (6-phthalazine carboxylic acid, 3,4-dihydro-1-(hydroxymethyl)-5,7-dimethyl-4-oxo-ethyl ester), their pharmaceutically equivalent salts, among others.

Hydrogel-based water-swelling polymers and non-gelling insoluble polymers can be used herein to adjust the release rate and bioavailability of the active drug ingredient with low water solubility. For example, ionic hydrogel polymers as well as non-ionic hydrogel polymers (e.g., non-ionic hydrophilic hydrogel polymers) can be used. As one example, a pharmaceutical-suitable homo-polymer hydrogel (such as a polymer polymerized from the same type of monomers without cross-linking to two or more different kinds of monomers, a polymer with the same kind of side chains, a non-copolymer) can be used. In one embodiment, the pharmaceutical composition may include about 4% to 80% by weight of the non-cross-linked, water-swelling homo-polymer.

Examples of the non-cross-linked, water-swelling homo-polymer include, but are not limited to, hydroxypropyl methylcellulose (HPMC, e.g., METHOCEL™, etc.), alginate, sodium alginate, cellulose hydrogel, polyvinylpyrrolidone, hydroxypropyl cellulose (HPC; e.g., KLUCEL™, etc.), nitrocellulose, hydroxypropyl ethylcellulose, hydroxypropyl butylcellulose, hydroxypropyl pentylcellulose, methyl cellulose, hydroxyethyl cellulose, alkyl celluloses, hydroxyalkyl celluloses, cellulose ethers, cellulose acetate, carboxymethyl cellulose, sodium carboxymethyl cellulose, calcium carboxymethyl cellulose, poly-hydroxyalkyl methacrylate, polymethacrylic acid, polymethylmethacrylate, poly vinyl alcohol, sodium polyacrylic acid, calcium polyacrylic acid, polyacrylic acid, acidic carboxy polymers, carboxypolymethylene, carboxyvinyl polymers, carboxymethylamide, polyoxyethyleneglycols, polyethylene oxide, and derivatives, their pharmaceutically equivalent salts, and mixtures thereof.

The non-gelling insoluble polymer used in the pharmaceutical composition may be a hydrophobic polymer that are water-insoluble at all pH ranges in order to help decrease the hydrophilicity of the water-swelling hydrogel polymer for preparing oral dosage forms of the hydrophobic drug.

The non-gelling insoluble polymer used in the pharmaceutical composition can be an enteric polymer where its solubility is pH-dependent. For example, an enteric polymer which is insoluble at acidic pH but soluble at higher pH range can be used. One example of an enteric polymer is EUDRAGIT® L100.

Examples of the non-gelling insoluble polymer include, but are not limited to, hydrophobic polymer (such as ethyl cellulose (e.g., ETHOCEL™, etc.), polymethyl acrylate polymer (e.g., EUDRAGIT® NE, EUDRAGIT® EC, etc.), anionic polymer, enteric polymer (e.g., EUDRAGIT® L, etc.), a pH-dependent insoluble polymer, and their derivatives, salts, and mixtures thereof. Additional examples of a water insoluble polymer include, but are not limited to, cellulose derivatives (e.g. cellulose acetate, etc.), polyvinyl acetate (e.g., KOLLICOAT™ SR30D from BASF), neutral copolymers based on ethyl acrylate and methylmethacrylate, copolymers of acrylic and methacrylic acid esters with quaternary ammonium groups (such as EUDRAGIT® NE, EUDRAGIT® RS, EUDRAGIT® RS30D, EUDRAGIT® RL, EUDRAGIT® RL30D, and the like), and their derivatives, salts, and mixtures thereof. In one embodiment, the pharmaceutical composition may include about 4% to 80% by weight of the non-gelling insoluble polymer.

Examples of the enteric polymer include, but are not limited to, esters of cellulose and its derivatives (such as cellulose acetate phthalate, hydroxypropyl methylcellulose phthalate, hydroxypropyl methylcellulose acetate succinate, and the like), polyvinyl acetate phthalate, pH-sensitive methacrylic acid-methamethacrylate copolymers and shellac, and their derivatives, salts, and mixtures thereof. Some commercially available enteric polymers that may be used are, for example, methacrylic acid copolymers sold under the trademark EUDRAGIT® (L100, S100, L30D) manufactured by Rhom Pharma, cellacefate (cellulose acetate phthalate) from Eastman Chemical Co., aquateric aqueous enteric polymer (cellulose acetate phthalate, for example, used as an aqueous dispersion) from FMC Corp., and AQOAT™ (hydroxypropyl methylcellulose acetate succinate or hypromellose acetate succinate, for example, used as aqueous dispersions) from Shin Etsu K.K., and other enteric coating materials. These enteric polymers may be used as a dry powder or an aqueous dispersion.

In one embodiment, a method for modifying the release rate of the hydrophobic drug using a water soluble hydrogel polymer is provided in order to obtain a controlled release drug formulation, such as a formulation that exhibits, for example, sustained release, constant release, extended release, or substantially zero-order release, etc. in its in vivo and in vitro drug dissolution and/or bioavailability profiles. The method may include adjusting the weight ratio of a water soluble hydrogel polymer and a non-gelling insoluble polymer in a pharmaceutical formulation at a weight ratio of about 1:10 to 10:1 to obtain a desired release rate profile. One example of a pharmaceutical formulation includes a water soluble hydrogel polymer and a non-gelling insoluble polymer at a weight ratio of about 4:1. Another example of a pharmaceutical formulation includes a water soluble hydrogel polymer and a non-gelling insoluble polymer at a weight ratio of about 1:4.

Further, a controlled release drug dissolution profile for the hydrophobic drug can be obtained. For example, a constant in vitro drug dissolution profile for the hydrophobic drug can be obtained. As another example, a zero degree release profile of the hydrophobic drug in a pharmaceutical formulation is obtained.

A method of administering a pharmaceutical composition containing a therapeutically-effective amount of a powder form of a hydrophobic drug is also provided. In one embodiment, the method includes administering to a mammal an effective amount of the pharmaceutical composition having a non-cross-linked, water-swelling homo-polymer and a non-gelling hydrophobic polymer combined at a weight ratio of about 1:10 to 10:1 and directly compressed with a therapeutically-effective amount of the hydrophobic drug.

The pharmaceutical formulation containing the hydrophobic drug can be prepared into an oral dosage form or a solid dosage form, such as a tablet, a capsule, a sachet etc., and any other therapeutically acceptable form. The hydrophobic drug can be prepared from a powder form, a micronized form, a granular form, a particle form, etc. The hydrophobic drug included in the formulation can be any desired therapeutically-effective dosage strength. In one embodiment, the hydrophobic drug is about 1% to 95% by weight of the pharmaceutical composition. For example, a pharmaceutical formulation for preparing cilostazol tablets may include about 100 mg, 200 mg, 300 mg, etc. of cilostazol.

Various approaches exist for preparing sustained or controlled release pharmaceutical formulations, such as various extended release formulations in tablet or capsule form. In general, wet granulation or dry granulation approaches can be used. For example, one method of forming delayed or sustained release formulations includes preparing drug-containing blended granules and compressing the granules into tablets. In addition, the tablet can be coated with a release-retarding coating. Alternatively, individual granules can be coated with such a release-retarding coating, and compressing these coated granules into a tablet. In addition to forming drug-containing granules, a dispersing agent can be used to improve solubility and dispersibility of a hydrophobic drug and preparing the hydrophobic drug in a dispersion form.

It is found that, even without forming into granules or dispersion, a therapeutically-effective amount of a hydrophobic drug can be surprisingly prepared into a pharmaceutical formulation through direct compression. For example, preparing a hydrophobic drug into a tablet in the presence of a water soluble hydrogel polymer and a release-rate-adjusting polymer through direct compression provides an efficient way to obtain a desired controlled release rate profile. In one embodiment, a therapeutically-effective amount of a powder form of a hydrophobic drug, a suitable amount of a powder form of a non-cross-linked, water-swelling homo-polymer; and a suitable amount of a powder form of a non-gelling insoluble polymer are combined and directly prepared into a desired oral dosage form, such as a tablet or a capsule. In addition, the hydrophobic drug containing oral dosage form can be further coated with an outer-layer coating. For example, prepared tablets or capsules can be film-coated, taste-mask coated, and/or enteric polymer coated, when necessary. The outer layer coating may also include the hydrophobic drug, binders, hydrophobic release modifying agents, lubricants, glidants enteric polymer, etc.

Optionally, the pharmaceutical formulation for preparing an oral dosage form of a hydrophobic drug may also include wetting agents, surfactants, emulsifiers, dispersing agents, defoamers, excipients, diluents, binders, release rate modifying agents, glidants, and lubricants, and mixtures thereof, etc. Any of the pharmaceutically acceptable or medicinally acceptable surfactants, emulsifiers, dispersing agents, dispersants, and defoamers can be used herein. For example, tween 80 (available form Fisher Scientific International), tween 20, tween 100, sodium lauryl sulfate, and others can be used to a concentration of no more than 50%, such as from about 0.1% to about 10%. One example of a wetting agent is a surfactant, such as SLS (sodium lauryl sulfate). For example, about 0.3% or about 0.5% of SLS can be used in the pharmaceutical formulation.

Further, the pharmaceutical formulation may include lubricants, blenders, anti-sticking agents, glidants, wetting agents, dyes, pigments, nonstick agents, dispersants, blenders, coating materials, and mixtures thereof, to be combined with the core of the pharmaceutical mixture. Examples of lubricants include, but are not limited to, stearic acid, glycerol monostearate, talc, calcium stearate, magnesium stearate, solid polyethylene glycols, sodium lauryl sulfate, inert silicon glass materials, colloidal silicon dioxide, and higher fatty acids and their alkali-metal and alkaline-earth-metal salts, among others. In addition, various excipients such as diluents, lubricants, dyes, etc., which are disclosed in Remington's Pharmaceutical Sciences, 1995 Edition, may be used to optimize the pharmaceutical composition. The amount of the lubricants and anti-sticking agents generally varies from about 0.5% to about 20% by weight of the pharmaceutical composition, such as from about 2.5% to about 10%. Examples that can be blended herein with the core of the pharmaceutical composition include magnesium stearate, silicon dioxide and talc to a final concentration of from about 1.0% to about 7.0% by weight. One example of a diluent is lactose.

### EXAMPLES

The following examples are intended to illustrate the invention without limiting the scope of the invention.

Suitable hydrogels include hydroxypropylmethl cellulose and the like. In addition, an effective amount of a non-toxic, pharmaceutically acceptable ionizable compound which is capable of modifying the release rate of the drug from the hydrogel can be included. The amount of the hydrogel used may be determined by preparing a series of tablets using varying amounts of hydrogel in combination with the hydrophobic drug, such as cilostazol. The release characteristics may be determined separately under various testing conditions, such as water/0.3% sodium lauryl sulfate, water/0.5% sodium lauryl sulfate, simulated gastric fluid (SGF, pH 1.2—without enzymes); simulated intestinal fluid (SIF, ph 7.5—without enzymes), pH 6.8 buffered conditions, etc.

The “paddle method” from United States Pharmacopeia (USP) XXII standards, which is incorporated by reference, may be used to determine the release characteristics of a given pharmaceutical formulation, the release curve for a particular drug can be modified to a zero-order release rate. Other standard methods from USP can also be used. The pharmaceutical composition may include about 1% to about 80% of a therapeutically amount of a hydrophobic drug and about 4% to about 80% of a water-swelling hydrogel polymer. The hydrophobic drug in the pharmaceutical composition may include cilostazol or its pharmaceutically equivalent salt thereof. One example of the water-swelling hydrogel polymer is hydroxypropyl methylcellulose.

According to one embodiment of the invention, an controlled release pharmaceutical composition is provided, including about 1% to about 80% of a therapeutically amount of cilostazol and about 4% to about 80% of a water-swelling hydrogel polymer. The controlled release pharmaceutical composition is formulated to obtain a constant release rate. For example, a controlled release pharmaceutical composition containing cilostazol and a water-swelling hydrogel polymer material at a zero order release rate is obtained. The water-swelling hydrogel polymer material may be hydroxypropyl methylcellulose.

According to another embodiment of the invention, a method of administering a pharmaceutical composition containing cilostazol is provided. The method includes administering to a mammal an effective amount of the pharmaceutical composition including about 1% to about 80% of a therapeutically amount of cilostazol and about 4% to about 80% of a water-swelling hydrogel polymer. One example of the water-swelling hydrogel polymer is hydroxypropyl methylcellulose.

According to another embodiment of the invention, the pharmaceutical composition may also include a surfactant, such as a hydrophilic surfactant or a hydrophobic surfactant. One example of a surfactant is about 0.01% to about 5% of sodium lauryl sulfate. In addition to the hydrogel and the active drug ingredient, the pharmaceutical composition may also include an inert solid diluent, such as lactose, dextrose, maltose, fructose, corn starch, rice starch and the like.

Other additives such as binding agents such as polyvinylpyrrolidoine, starch, gelatin, microcrystalline cellulose and the like may be added to the tablet formulation. Further, it is contemplated that coloring agents, stabilizers, lubricants such as stearic acid, palmitic acid, magnesium stearate, and the like may be added to the tabletting composition in amounts which are determined to produce desired in vivo and in vitro drug release performance. Oral dosage forms, such as tablets and gels, may be made using conventional process in appropriate sizes.

### Example 1

Cilostazol 150 mg extended release tablets were prepared. Each tablet includes about 150 mg of cilostazol, 11.7% by weight of hydroxypropyl methylcellulose, 1.7% by weight of sodium lauryl sulfate, 33% by weight of lactose, and about 3.3% by weight of glycerol monostearate. The tablets are prepared through direct compression using a rotary press.

### Example 2

Cilostazol extended release tablets having about 150 mg of cilostazol, 18.3% by weight of hydroxypropyl methylcellulose, 1.7% by weight of sodium lauryl sulfate, 26.7% by weight of lactose, and about 3.3% by weight of glycerol monstearate were prepared.

### Example 3

Cilostazol extended release tablets having about 150 mg of cilostazol, 10% by weight of hydroxypropyl methylcellulose, 36.7% by weight of lactose, and about 3.3% by weight of glycerol monostearate were prepared.

### Example 4

Cilostazol extended release tablets having about 150 mg of cilostazol, 16.7% by weight of hydroxypropyl methylcellulose, 30.0% by weight of lactose, and about 3.3% by weight of stearic acid were prepared.

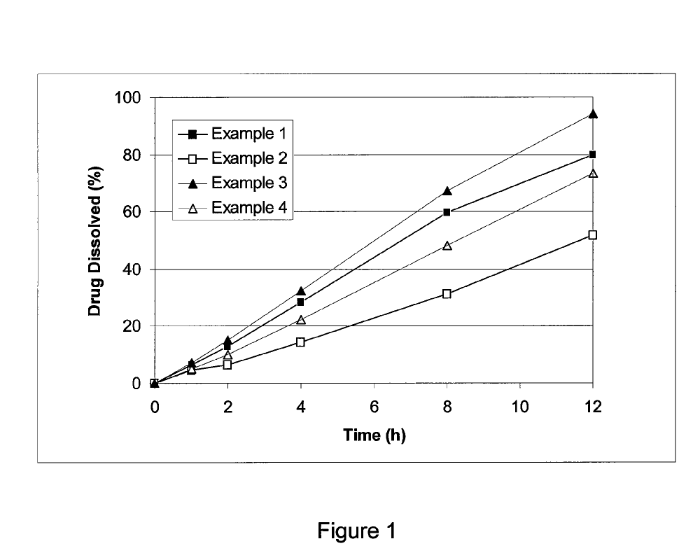

FIG. 1 illustrates the in vitro dissolution profiles of representative oral dosage forms of cilostazol prepared as described in Examples 1-4 in accordance with one or more embodiments of the invention. All of which exhibit a constant release rate. The in vitro dissolution profiles of all the tablets prepared according to Examples 1-4 exhibit a zero order release rate, suitable to be used as controlled release or extended release oral dosage forms of cilostazol.

### Example 5

Cilostazol controlled release tablets having about 150 mg of cilostazol, hydroxypropyl methylcellulose, a non-gelling insoluble polymer, a diluent, and a lubricant were prepared (300 mg total weight for each tablet).

Dissolution Profile of Example 5

| | % released | % released |
|---|---|---|
| Time, hours | under SGF/0.5% SLS | under SIF/0.5% SLS |
| 0 | 0 | 0 |
| 1 | 9.6 | 9.12 |
| 2 | 23.6 | 14.16 |
| 4 | 30.4 | 34.25 |
| 8 | 64.6 | 71.5 |
| 12 | 95.7 | 99.22 |

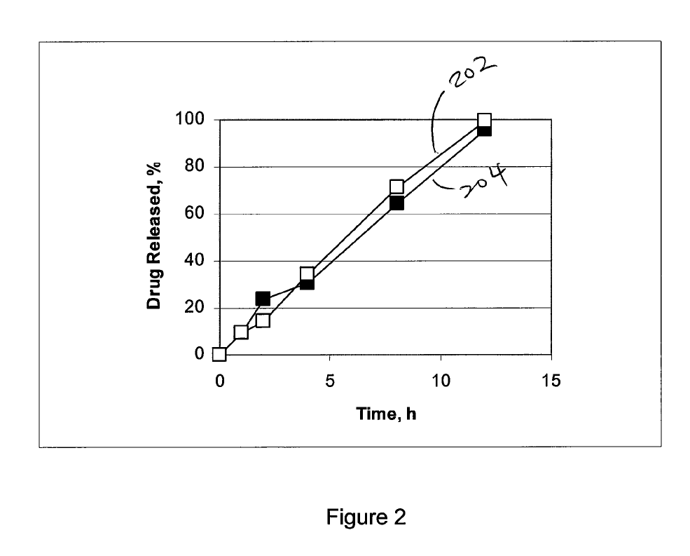

FIG. 2 illustrates the in vitro dissolution profiles of representative oral dosage forms of cilostazol prepared as described in Example 5 and tested using under SIF and SGF condition, as shown as lines 202 and 204, respectively, according to the procedure described in United States Pharmacopeia (USP), Apparatus 2, at a paddle speed of about 50 rpm, and in the presence of about 0.5% of sodium lauryl sulfate. The in vitro dissolution profiles of all the tablets prepared according to Example 5 exhibit a zero order release rate under both SIF and SGF conditions, suitable to be used as controlled release or extended release oral dosage forms of cilostazol.

### Example 6

Cilostazol controlled release tablets having about 300 mg of cilostazol, hydroxypropyl methylcellulose, a hydrophobic polymer, a diluent, and a lubricant were prepared (total weight: 600 mg each tablet).

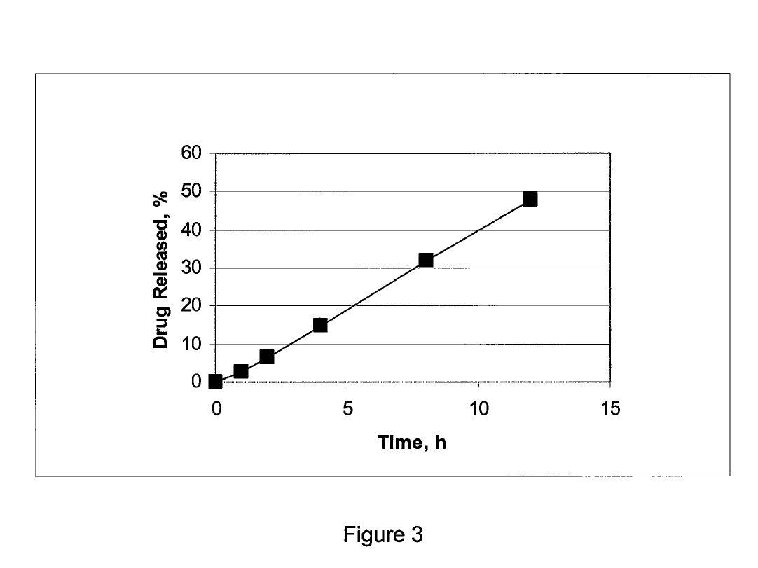

FIG. 3 illustrates the in vitro dissolution profiles of representative oral dosage forms of cilostazol prepared as described in Example 6 and tested according to the procedure described in United States Pharmacopeia at a paddle speed of about 50 rpm and in the presence of about 0.3% of sodium lauryl sulfate. The in vitro dissolution profiles of all the tablets prepared according to Example 6 exhibit a zero order release rate, suitable to be used as controlled release or extended release oral dosage forms of cilostazol.

Dissolution Profile of Example 6

| Time, hours | % released |
|---|---|
| 0 | 0 |
| 1 | 2.71 |
| 2 | 6.31 |
| 4 | 14.83 |
| 8 | 31.89 |
| 12 | 47.67 |

### Example 7

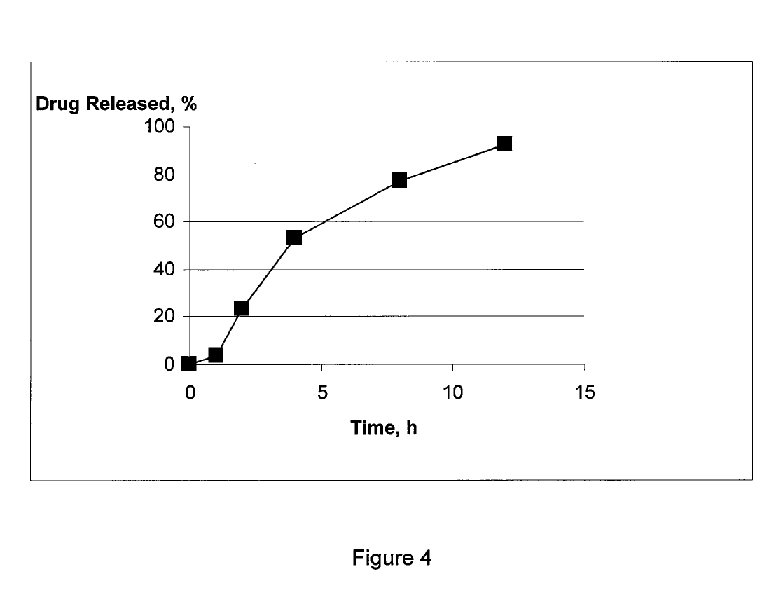

Cilostazol controlled release tablets having about 100 mg of cilostazol, hydroxypropyl methylcellulose, a hydrophobic polymer, a diluent, and a lubricant were prepared. FIG. 4 illustrates the in vitro dissolution profiles of representative oral dosage forms of cilostazol prepared as described in Example 7 and tested according to the procedure described in United States Pharmacopeia at a paddle speed of about 50 rpm and in the presence of about 0.3% of sodium lauryl sulfate. The in vitro dissolution profiles of all the tablets prepared according to Example 7 exhibit controlled release of cilostazol.

Dissolution Profile of Example 7

| Time, hour | % released |
|---|---|
| 0 | 0 |
| 1 | 3.63 |
| 2 | 23.56 |
| 4 | 53.24 |
| 8 | 77.51 |
| 12 | 92.35 |

### Example 8

Cilostazol controlled release tablets having about 150 mg of cilostazol, sodium alginate, a hydrophobic polymer, a diluent, and a lubricant were prepared (total weight: 300 mg each tablet).

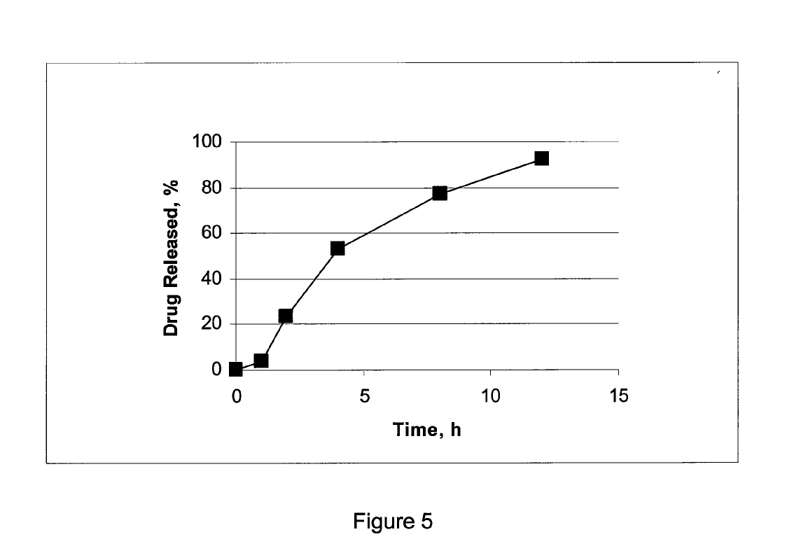

FIG. 5 illustrates the in vitro dissolution profiles of representative oral dosage forms of cilostazol prepared as described in Example 8 and tested according to the procedure described in United States Pharmacopeia at a paddle speed of about 50 rpm and in the presence of about 0.3% of sodium lauryl sulfate. The in vitro dissolution profiles of all the tablets prepared according to Example 8 exhibit controlled release of cilostazol.

Dissolution Profile of Example 8

| Time, hours | % released |
|---|---|
| 0 | 0 |
| 1 | 27.8 |
| 2 | 59.2 |
| 4 | 90.9 |
| 8 | 96.5 |
| 12 | 96.6 |

### Example 9

Dissolution Profile of Example 9

| Time, h | % released |
|---|---|
| 0 | 0 |
| 1 | 8.8 |
| 2 | 20.9 |
| 4 | 33.8 |
| 8 | 47.4 |
| 12 | 60.12 |

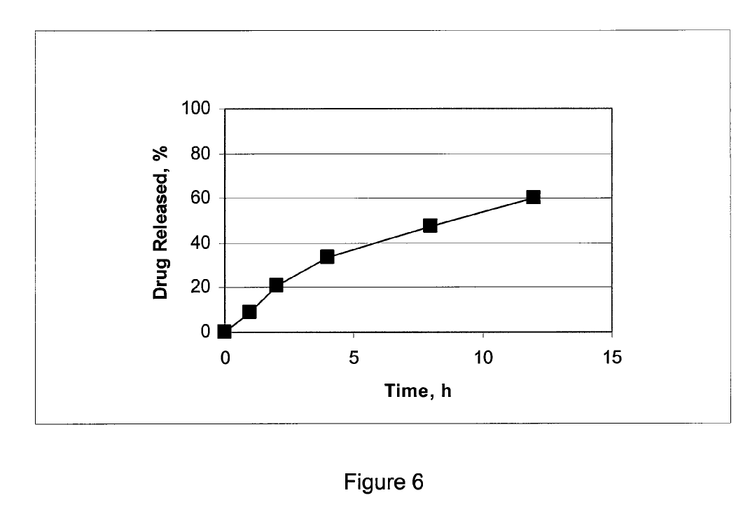

Cilostazol controlled release tablets having about 150 mg of cilostazol, EUDRAGIT® NE, a diluent, and a lubricant were prepared (total weight: 300 mg each tablet). FIG. 6 illustrates the in vitro dissolution profiles of representative oral dosage forms of cilostazol prepared as described in Example 9 and tested using according to the procedure described in United States Pharmacopeia at a paddle speed of about 50 rpm and in the presence of about 0.3% of sodium lauryl sulfate. The in vitro dissolution profiles of all the tablets prepared according to Example 9 exhibit sustained release of cilostazol.

### Example 10

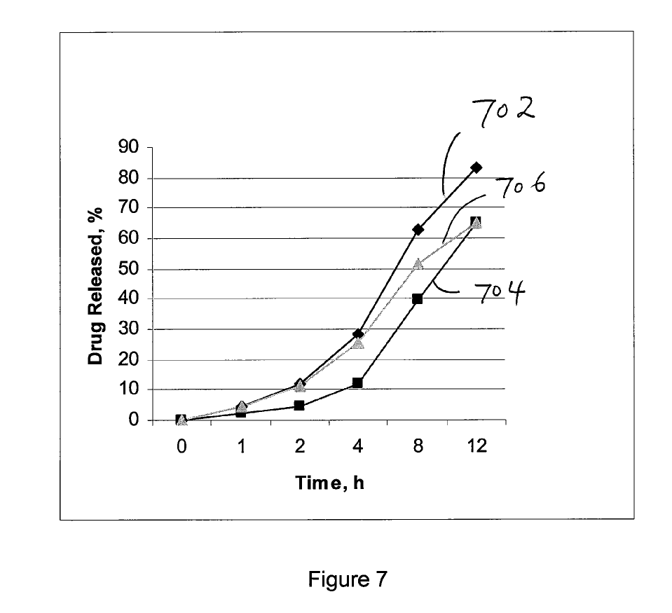

Cilostazol controlled release tablets having about 100 mg of cilostazol, a water-swelling hydrogel homo-polymer, a non-gelling insoluble polymer, a diluent, and a lubricant were prepared (total weight: 200 mg each tablet). FIG. 7 illustrates the in vitro dissolution profiles of representative oral dosage forms of cilostazol prepared as described in Example 10 and tested according to the procedure described in United States Pharmacopeia at a paddle speed of about 50 rpm and in the presence of about 0.3% of sodium lauryl sulfate.

Dissolution profiles of Example 10

| Time | % released | % released | % released |
|---|---|---|---|
| hours | Line 702 | Line 704 | Line 706 |
| 0 | 0 | 0 | 0 |
| 1 | 4.4 | 2.5 | 4.8 |
| 2 | 11.7 | 4.6 | 11.6 |
| 4 | 28.5 | 11.7 | 25.7 |
| 8 | 62.8 | 39.5 | 51.5 |
| 12 | 83.4 | 65.2 | 65.1 |

As shown in FIG. 7, the in vitro dissolution profiles of all the tablets prepared according to Example 10 exhibit controlled release of cilostazol. Lines 702 and 704 represent dissolution profiles of tablets with different weight ratios of the water-swelling hydrogel homo-polymer and the non-gelling insoluble polymer (about 4:1 and 1:4, respectively). Lines 702 and 706 represent dissolution profiles of tablets using the same water-swelling hydrogel homo-polymer combined with different non-gelling insoluble polymers at the same weight ratio of about 4:1.

While the foregoing is directed to embodiments of the present invention, other and further embodiments of the invention may be devised without departing from the basic scope thereof, and the scope thereof is determined by the claims that follow.

What is claimed is:

1. A pharmaceutical composition, comprising:
a powder form of a non-cross-linked, water-swelling homo-polymer; and
a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of a hydrophobic drug.

2. The pharmaceutical composition of claim 1, further comprises a wetting agent.

3. The pharmaceutical composition of claim 2, wherein the wetting agent is a surfactant.

4. The pharmaceutical composition of claim 1, wherein the hydrophobic drug is about 1% to 95% by weight of the pharmaceutical composition.

5. The pharmaceutical composition of claim 1, wherein the hydrophobic drug is cilostazol or its pharmaceutically equivalent salts thereof.

6. The pharmaceutical composition of claim 1, wherein the hydrophobic drug is doxazocin mesylate or its pharmaceutically equivalent salts thereof.

7. The pharmaceutical composition of claim 1, wherein the non-cross-linked, water-swelling homo-polymer is about 4% to 80% by weight of the pharmaceutical composition.

8. The pharmaceutical composition of claim 1, wherein the non-cross-linked, water-swelling homo-polymer is a non-ionic polymer.

9. The pharmaceutical composition of claim 1, wherein the non-cross-linked, water-swelling homo-polymer is an ionic polymer.

10. The pharmaceutical composition of claim 1, wherein the non-gelling insoluble polymer is about 4% to 80% by weight of the pharmaceutical composition.

11. The pharmaceutical composition of claim 1, wherein the non-cross-linked, water-swelling homo-polymer is selected from the group consisting of hydroxypropyl methylcellulose, alginate, sodium alginate, hydroxypropyl cellulose, cellulose hydrogel, and combinations thereof.

12. The pharmaceutical composition of claim 1, wherein the non-gelling insoluble polymer is a hydrophobic polymer.

13. The pharmaceutical composition of claim 1, wherein the non-gelling insoluble polymer is an anionic polymer.

14. The pharmaceutical composition of claim 1, wherein the non-gelling insoluble polymer is selected from the group consisting of ethyl cellulose, polymethyl acrylate polymer, hydrophobic water-insoluble polymer, anionic water-insoluble polymer, enteric water-insoluble polymer, pH-dependent water-insoluble polymer, cellulose acetate, polyvinyl acetate, and combinations thereof.

15. A controlled release pharmaceutical composition, comprising:
a powder form of a non-cross-linked, water-swelling homo-polymer; and
a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of cilostazol at about 1% to 95% by weight of the pharmaceutical composition.

16. The controlled release pharmaceutical composition of claim 15, wherein the dissolution of cilostazol is at a zero order release rate.

17. The controlled release pharmaceutical composition of claim 15, wherein the non-cross-linked, water-swelling homo-polymer is selected from the group consisting of hydroxypropyl methylcellulose, alginate, sodium alginate, hydroxypropyl cellulose, cellulose hydrogel, and combinations thereof.

18. The controlled release pharmaceutical composition of claim 15, wherein the non-cross-linked, water-swelling homo-polymer is hydroxypropyl methylcellulose.

19. The controlled release pharmaceutical composition of claim 15, wherein the non-cross-linked, water-swelling homo-polymer is a non-ionic polymer.

20. The pharmaceutical composition of claim 15, wherein the non-cross-linked, water-swelling homo-polymer is an ionic polymer.

21. The pharmaceutical composition of claim 15, wherein the non-gelling insoluble polymer is selected from the group consisting of ethyl cellulose, polymethyl acrylate polymer, hydrophobic water-insoluble polymer, anionic water-insoluble polymer, enteric water-insoluble polymer, pH-dependent water-insoluble polymer, cellulose acetate, polyvinyl acetate, and combinations thereof.

22. The pharmaceutical composition of claim 15, further comprises a wetting agent.

23. A controlled release pharmaceutical composition, comprising:
a powder form of a non-cross-linked, water-swelling homo-polymer; and
a powder form of a non-gelling insoluble polymer, where the non-cross-linked, water-swelling homo-polymer and the non-gelling insoluble polymer are combined at a weight ratio of about 1:10 to 10:1 and directly compressed with a therapeutically-effective amount of a powder form of doxazocin mesylate at about 1% to about 95% by weight of the pharmaceutical composition.

24. The controlled release pharmaceutical composition of claim 23, wherein the non-cross-linked, water-swelling homo-polymer is selected from the group consisting of hydroxypropyl methylcellulose, alginate, sodium alginate, hydroxypropyl cellulose, cellulose hydrogel, and combinations thereof.

25. The pharmaceutical composition of claim 23, wherein the non-gelling insoluble polymer is selected from the group consisting of ethyl cellulose, polymethyl acrylate polymer, hydrophobic water-insoluble polymer, anionic water-insoluble polymer, enteric water-insoluble polymer, pH-dependent water-insoluble polymer, cellulose acetate, polyvinyl acetate, and combinations thereof.

26. A method of administering a pharmaceutical composition containing a hydrophobic drug, comprising:
administering to a mammal an effective amount of the pharmaceutical composition comprising a power form of a non-cross-linked, water-swelling homo-polymer and a powder form of a non-gelling hydrophobic polymer, where the non-cross-linked, water-swelling homo-polymer and the non-gelling insoluble polymer are combined at a weight ratio of about 1:10 to 10:1 and directly compressed with a therapeutically-effective amount of a powder form of the hydrophobic drug.

27. The method of claim 26, wherein the hydrophobic drug is selected from the group consisting of cilostazol, doxazocin mesylate, and their pharmaceutically equivalent salts thereof.
